(19) United States
(12) Patent Application Publication
(10) Pub. No.: US 2021/0393444 A1
(43) Pub. Date: Dec. 23, 2021

(54) FRICTION REDUCING DEVICES AND ORTHOPEDIC FOOT INSERTS

(71) Applicants: Samuel Scheinberg, Tualatin, OR (US); Cheryl Scheinberg, Wilsonville, OR (US)

(72) Inventors: Samuel Scheinberg, Tualatin, OR (US); Cheryl Scheinberg, Wilsonville, OR (US)

(21) Appl. No.: 17/464,875

(22) Filed: Sep. 2, 2021

Related U.S. Application Data
(63) Continuation-in-part of application No. 15/993,349, filed on May 30, 2018.
(60) Provisional application No. 62/518,440, filed on Jun. 12, 2017.

(51) Int. Cl. A61F 13/06; A61F 13/02; A43B 7/14; A43B 7/28

(52) U.S. Cl. CPC A61F 13/067; A61F 13/0276; A61F 2013/00331; A43B 7/28; A43B 7/1455

(57) ABSTRACT

Disclosure herein are protective bandage and methods of use and manufacture thereof. The protective bandages can include first and second flexible films and a shear-reducing element. The first flexible film may form a contact layer having a first side opposite a second side. The second flexible film may form a dome attached to and extending over a portion of the first side of the contact layer. The dome may have a height at least partially defined by a side wall and a top portion of the second flexible film. The contact layer may extend beneath the dome and the top portion being freely movable along the first side of the contact layer through a distance related to the height. The shear-reducing element is located inside the hollow dome.

### CROSS-REFERENCE TO RELATED APPLICATIONS

This application is a continuation-in-part of U.S. patent application Ser. No. 15/993,349, filed May 30, 2018, which application claims priority to U.S. Provisional Patent Application Ser. No. 62/518,440, filed Jun. 12, 2017, the contents of which are hereby incorporated in their entirety.

This application is related to U.S. patent application Ser. No. 10/672,731, filed Sep. 25, 2003, and granted as U.S. Pat. No. 7,087,806 on Aug. 8, 2006, which is a continuation-in-part of U.S. patent application Ser. No. 10/637,429, filed on Aug. 8, 2003, the contents of each which is hereby incorporated in its entirety.

### FIELD OF THE DISCLOSURE

The present subject matter relates to friction reducing devices and orthopedic foot inserts. More specifically, the present disclosure relates to relieving foot discomfort by providing a comfort-promoting insert that can be utilized within footwear.

### BACKGROUND

It is well known that a person can experience great discomfort as a result of standing for lengthy times, particularly when wearing dress shoes, which often have hard, relatively incompressible heels and soles and which may lack significant cushioning in their insoles. When forced to stand relatively still or to stand for lengthy times on an unyielding surface such as a concrete or hardwood floor, and even when standing on a carpeted floor, a person's foot may stay practically motionless within the confines of a shoe. The nearly constant pressure and the effects on one's foot of shear stress, caused by the interior surfaces of a shoe tending to pull the skin of the bottom and sides of the person's foot in one direction or another, can result in a significant amount of pain and tissue break-down. In addition, ill-fitting shoes that don't support the heels from expanding sideways under pressure, in conjunction with skin that is genetically dry or dry from metabolic conditions associated with aging and diabetes can lead to extremely painful skin fissures most commonly seen in the heel area. Shear and friction forces exacerbate these cracks, increase pain, and prolong healing.

Many shoes, particularly athletic shoes, incorporate padded and appropriately-shaped insoles which may be of significant benefit in preventing discomfort and pain. Most dress shoes, including women's high-heeled shoes, however, have relatively hard and inflexible insoles which may not be well shaped to conform to a person's foot. Dress shoes for men commonly have stiff unyielding rear quarters and front vamps that do not match the wearer's anatomy. These portions of the shoe may be too tight, excessively compressing the wearer's heel or forefoot, or too loose, allowing the foot soft tissue to expand over time and flatten within the shoe. The former condition leads to skin breakdown and blister formation, the latter condition may result in skin fissuring and cracking. The insoles of men's and women's dress shoes often are not shaped to conform to the arches of a person's foot, and sometimes are not even smooth. They may not include sufficient padding to prevent the wearer from feeling structural parts of the shoe, such as nails attaching a heel to a shoe sole. Standing for lengthy times in such dress shoes may often be necessary, however, for a person to present a socially desirable appearance or an appropriate business appearance.

While it may be impossible to avoid wearing shoes that are uncomfortable, it is desired to provide a remedy for the situation so that a person can dress in what may be socially-required footwear while standing for a considerable amount of time without pain or discomfort. In order to remedy that situation, it has long been known that various types of scientifically-shaped and cushioned inserts, some of which are specifically designed for a person's foot, can be worn within a shoe to improve comfort. They may also require some careful selection and may need to be cut to a particular shape to fit in a particular shoe. Some such inserts can be fashioned by specialists, as by molding foam materials to fit a particular person's foot and shoe. Most such inserts, however, are relatively expensive.

What is needed, then, is a comfortable yet simple and inexpensive insert that can be worn in a person's shoe to prevent or provide relief from discomfort and pain. Preferably, such an insert should not require extensive or skillful measurement and fitting to be effective and to maintain efficacy during extended use.

### BRIEF DESCRIPTION OF THE FIGURES

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of a bandage which is one preferred embodiment of the present invention.

FIG. 2 is a top plan view of the bandage shown in FIG. 1.

FIG. 3 is a section view taken on line 3-3 of FIG. 2 with thicknesses greatly exaggerated.

FIG. 4 is a section view taken on line 4-4 of FIG. 2 with thicknesses greatly exaggerated.

FIG. 5 is a partially cutaway sectional detail view of the dome top layer of the bandage, taken in the direction indicated by line 4-4 of FIG. 2.

FIG. 6 is a view showing the bandage shown in FIG. 1 in place on a person's ankle.

FIG. 7 is a perspective view of the bandage shown in FIG. 1, in place on a person's toe.

FIG. 8A is a flow diagram showing steps involved in manufacturing a dome top portion for the bandage shown in FIG. 1.

FIG. 8B is a flow diagram showing steps involved in assembling a dome base portion for the bandage shown in FIG. 1.

FIG. 9 is a flow diagram showing further steps according to the method of the present invention for manufacturing the bandage shown in FIG. 1.

FIG. 10 is a top plan view of a bandage which is an alternative embodiment of the invention.

FIG. 11 is a top plan view of a bandage which is another alternative embodiment of the present invention.

FIG. 12 is a partially cutaway isometric view taken from the bottom side of a bandage which is another alternative embodiment of the present invention.

FIG. 13 is a perspective view from the upper front side of a heel counter insert for use in a sports shoe, including a friction-reducing device according to the present invention.

FIG. 14 is a sectional view of the heel counter insert shown in FIG. 13, taken along line 14-14 of FIG. 13.

FIG. 15 is a perspective view of an insole, including a friction-reducing device according to the present invention.

FIG. 16 is a sectional view of the insole shown in FIG. 15, taken along line 16-16 of FIG. 15.

FIG. 17 is a side elevational view of a sports shoe incorporating friction-reducing devices according to the present invention.

FIG. 18 is a sectional view taken along line 18-18 of FIG. 17.

FIG. 19A is an isometric view of materials to be incorporated in a comfort insert for a person's footwear.

FIG. 19B is a detail view of a portion of a layer of netting material that may be used as one component of the comfort insert.

FIG. 19C is a view similar to that of FIG. 19A showing a partially assembled comfort insert.

FIG. 19D is an elevational view of a completed comfort insert, as it appears prior to use.

FIG. 20 is a side elevational view of a shoe, showing various positions where the comfort insert shown in FIG. 19D might be utilized.

FIG. 21 is a sectional view taken in a longitudinal vertical plane in a heel portion of the shoe shown in FIG. 20, showing a portion of a person's heel, along with the comfort insert in place within the shoe.

FIG. 22 is a sectional view taken in the direction of line 22-22 in FIG. 20.

FIG. 23 is a detail view at an enlarged scale, showing a portion of the sectional view in FIG. 21 of the comfort insert in use.

FIG. 24 is a view a bandage and use thereof in accordance with at least one embodiment of this disclosure.

FIG. 25 is a plan view of the bandage in FIG. 24 in accordance with at least one embodiment of this disclosure.

FIG. 26 is a view a bandage and use thereof in accordance with at least one embodiment of this disclosure.

FIG. 27 is a view a bandage and use thereof in accordance with at least one embodiment of this disclosure.

FIG. 28 is a plan view of the bandage in FIGS. 26 and 27 in accordance with at least one embodiment of this disclosure.

### DETAILED DESCRIPTION

As disclosed herein, protective bandages and comfort inserts may be used to relieve pain and/or provide protection for wounds to promote healing. In addition, the protective bandages and comfort inserts, can be used to in a preventative manner to provide friction reduction between a skin layer and a surface of a garment or other object in contact with the skin layer. For example, the protective bandages and comfort inserts can be placed in a shoe, on a surface of a wheelchair, beddings, etc. to provide friction reduction to reduce the risk of blisters, bedsores, etc. from forming. As disclosed herein, the term protective bandage(s) may be used interchangeably with comfort insert(s).

The protective bandages disclosed herein may include flexible films that are used to form contact layers and dome shaped structures attached to the contact layers. A shear-reducing element may be located within the dome. The shear-reducing element may include medications that flow through a skin contacting layer, which may be a permeable membrane. The shear-reducing element may include a plurality of layers of a thin plastic film material. The thin plastic film material may be folded to form the plurality of layers. The shear-reducing element may includes a lubricant located in between the plurality of layers. The lubricant may also be present when the plurality of layers are not present. An example of a shear-reducing element includes a quantity of air between the plurality of layers and/or inside the dome. The dome material may be formed of an open mesh netting material.

The above discussion is intended to provide an overview of subject matter of the present patent application. It is not intended to provide an exclusive or exhaustive explanation of the invention. The description below is included to provide further information about the present patent application.

Referring to FIGS. 1-4 of the drawings which form a part of the disclosure herein, an elliptical bandage 14 which is one preferred embodiment of the present invention includes a skin contact layer 16 of flexible film material to which a layer 18 of an adhesive material is adhered. The layer 18 of adhesive material is protected by an easily removable liner 20 divided into two separate halves by a cut 22 extending across the liner 20 to allow the halves of the liner 20 to be removed separately during application of the bandage 14 to a person's skin. Alternatively, one part of the liner 20 can overlap the other along the location of the cut 22, with the overlying portion including a folded-back margin flap 23 shown in broken line, to facilitate removal of the liner 20 from the skin contact layer 20 to apply the bandage. As one preferred size, the bandage 14 may have a length 24 of 66.67 mm (2.625 in.) and a width 26 of 38.1 mm (1.5 in.).

A dome 28 is centrally located on and adhesively attached to the skin contact layer 16. The dome 28 includes a top portion 30 and a circumferential side wall portion 32 interconnecting the top portion with the skin contact layer 16 and with intermediate portions of the bandage 14, as will be described in more detail presently. The dome 28 is of a thin, strong, flexible, film material, and its top portion 30 is free to move parallel with the skin contact layer 16 in any direction from a neutral position, as limited principally by the height 33 of the side wall portion 32 of the dome. The dome may have a diameter 29 of 25.15 mm (0.990 in.), and the top portion 30 may have a diameter 31 of 22.86 mm (0.0900 in.), for example, and the dome 28 may have a height 33 of 4.76 mm (0.1875 in.).

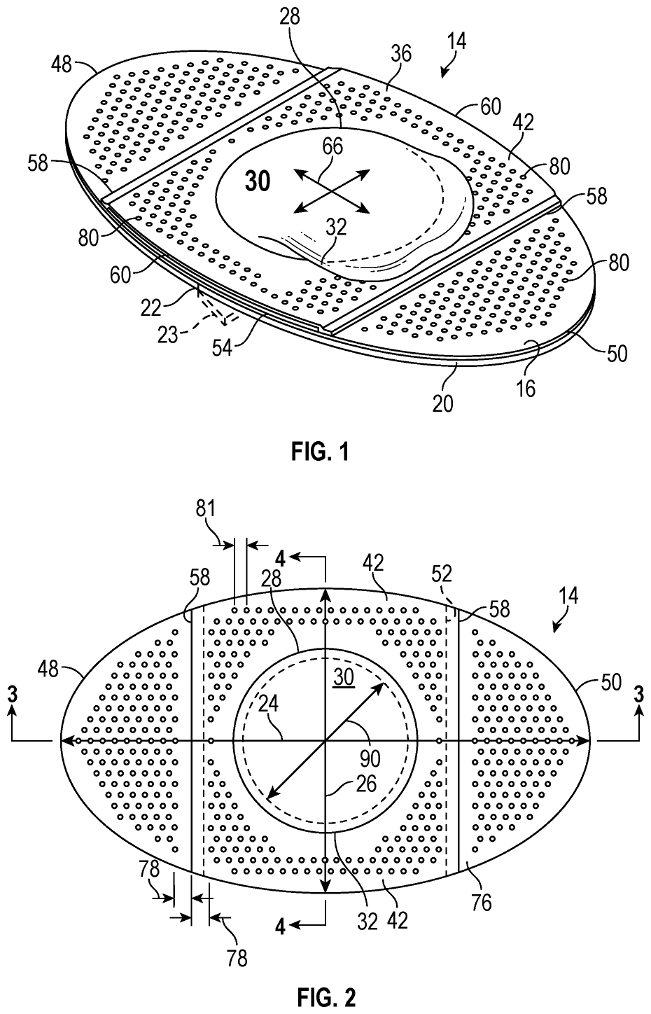

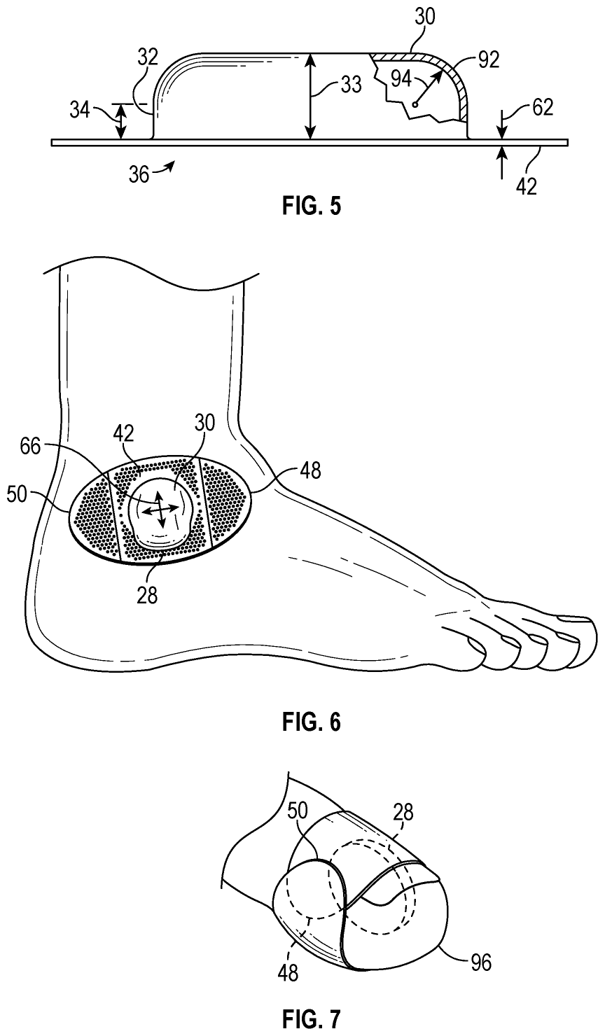

As shown in FIGS. 2-5, in the preferred embodiment of the bandage 14, the dome 28 includes a dome top layer 36 and a dome base layer 38. The dome base layer 38 is fastened to a first, or upper, side of the skin contact layer 16, for example being adhesively attached by a layer of adhesive material 40 that is coextensive with the dome base layer 38. As used herein, the term adhesively attached should be understood to include the use of various mechanisms such as thermal fusion, ultrasonic fusion, and chemical fusion to interconnect layers of the bandage 14, as well as the use of layers of adhesive materials such as the particular pressure sensitive adhesives described herein in detail.

The dome top layer 36 includes the top portion 30, the side wall portion 32, and a skirt portion 42 that is generally flat and which surrounds and extends radially outward in all directions from the base of the side wall portion 32. The skirt portion 42 is adhesively attached to the dome base layer 38 and the skin contact layer 16, as by a layer 44 of adhesive material. The layer 44 of adhesive material is coextensive with the skirt portion 42 but does not extend onto the side wall portion 32 or the top portion 30 of the dome 28.

The skin contact layer 16 is larger than either the dome base layer 38 or the dome top layer 36 and preferably has an elliptical or other elongated oval shape, extending away from the dome 28 in both of a pair of opposite directions to opposite ends 48 and 50. Other shapes could also be useful for use of the bandage 14 in a particular place.

The dome base layer 38 has a pair of opposite and parallel straight margins 52 extending transversely across the skin contact layer 16, while arcuate opposite ends 54 of the dome base layer 38 coincide with portions of the side margins 56 of the skin contact layer 16. The skirt portion 42 of the dome top layer 36 similarly has a pair of parallel straight margins 58 and a pair of arcuate opposite ends 60 which also coincide with portions of the side margins 56 of the skin contact layer 16.

The straight margins 58 of the dome top layer 36 are separated from each other by a distance 72 that is somewhat greater than the distance 70 between the parallel straight margins 52 of the dome base layer 38, so that each straight margin 58 extends beyond the adjacent straight margin 52 toward a respective nearer one of the opposite ends 48 and 50 of the skin contact layer 16 on either end of the bandage 14. For example, the width 70 of the dome base layer 38 between its straight margins 52 is preferably 28.6 mm (1.125 in.), while the width 72 of the dome top layer 36 may be 31.75 mm (1.25 in.), so that a portion of the skirt portion 42 attached to the skin contact layer 16 overlaps the dome base layer 38 on each side by a width 74 of about 1.6 mm (0.0625 in) beyond the straight margin 52. The adhesive layer 44 thus attaches the dome top layer 36 both to the dome base layer 38, in an area surrounding the dome 28, and to the first or upper side of the skin contact layer 16, in narrow areas between the straight margins 52 and 58, between the dome 28 and each of the opposite ends 48 and 50 of the skin contact layer 16. This arrangement with the margins 58 of the skirt portion 42 overlapping beyond the margins 52 provides a smooth contour of the bandage 14 in the connection of the dome top layer 36 to the skin contact layer 16, and adds to security of the connection of the dome 28 to the skin contact layer 16.

Since the adhesive layer 44 is present only on the skirt portion 42 of the dome top layer 36, the top portion 30 of the dome is free from the opposing upper surface of the dome base layer 38. The top portion 30 thus can move parallel with and along the upper surface of the dome base layer 38 in any direction in which it is urged, to the extent that it is permitted to move by the height 33 of the side wall portion 32. The thin film material of the dome top layer 36 and dome base layer 38 is a flexible and strong membrane, and has a low enough coefficient of friction, when rubbing against surfaces of similar material, that there is significantly less friction between the top portion 30 of the dome and the dome base layer 38 than is likely between, for example, a person's skin and a sock pressed against the skin by the inside of a shoe. The dome 28 may also include a lubricant or other low friction material located therewithin to reduce friction between the dome top layer 36 and the skin contact layer 16.

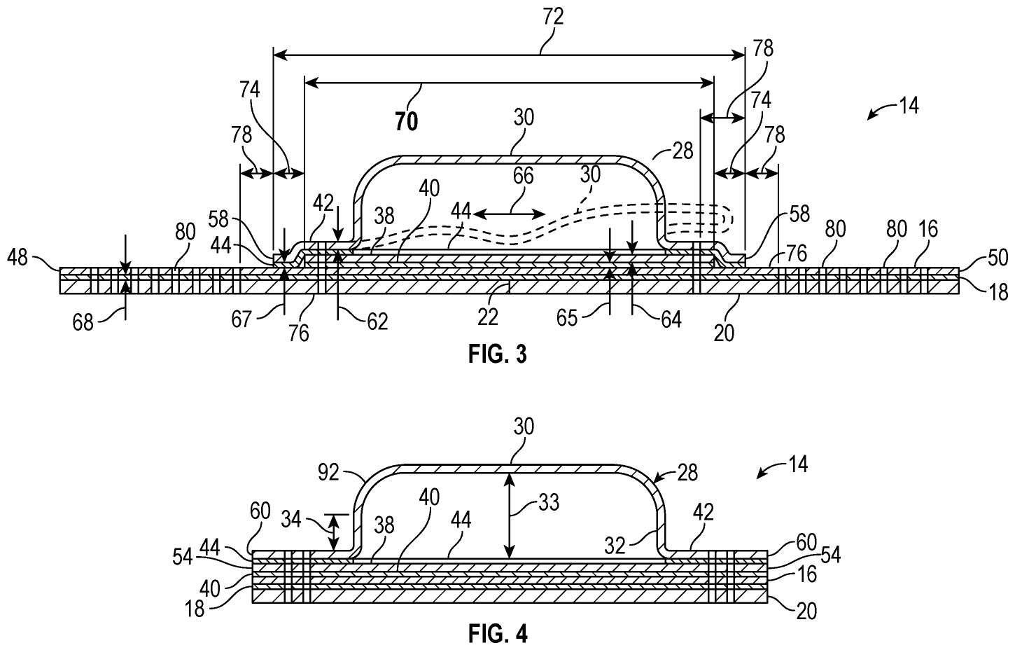

The film of which the dome top layer 36 may also be somewhat pervious to gas, so that the dome 28 can inflate, deflate, or collapse, according to the design of a particular dome, and particular material used as the dome top layer 36. The top portion 30 can contact and move in any direction along the dome base layer 38 (or the skin contact layer 16, should there be no dome base layer 38), as indicated by the arrow 66. Thus, while the dome 28 is shown in a neutral position in FIGS. 2-5, it is shown in FIG. 1 in a collapsed condition and offset toward the end 50 of the base layer 16, as also indicated in broken line in FIG. 3.

In order to protect a portion of a person's skin exposed to pressure or rubbing without adding to potential irritation, the entire bandage 14 is flexible and preferably as thin as practical consistent with sufficient strength. Accordingly, the dome top layer 36 may be of polyethylene film having a thickness 62 of about 25 microns (1 mil), and the dome base layer 38 is preferably of similar film material also having a thickness 64 of about 25 microns (1 mil). In such a small thickness, the polyethylene is amply flexible yet strong enough to withstand the usual forces to be encountered. A suitable polyethylene film is available in such a thickness from Quality Extrusion of Mankato, Minn., as its QCE 5% EVA type A polyethylene film.

Preferably, the adhesive layers 40 and 44 respectively interconnecting the dome base layer 38 with the skin contact layer 16 and interconnecting the skirt portion 42 of the dome top layer 36 with the dome base layer 38 and the skin contact layer 16 have similar thicknesses 65 and 67 of about 25 microns (1 mil). One suitable transfer adhesive is a non-sensitizing medical grade, biocompatible transfer adhesive, available from Tyco Adhesives of Norwood, Mass., as its “TR 2295C Medical Grade Transfer Adhesive,” in the form of a coiled transfer tape, a web of adhesive material carried on a backing or liner that is relatively easily removable after the adhesive is mated with the polyethylene film material of either the dome top layer 36 or the dome base layer 38.

A suitable material for the skin contact layer 16 is a polyurethane film having a thickness 68 of about 50 microns (2 mils). Such a polyurethane film is suitably strong and flexible and also is slightly more elastic than polyethylene, and thus is more able to conform to a person's skin as the skin stretches and relaxes during movement, than the polyethylene material preferred for the dome top layer 36 and base layer 38.

The film used to construct the skin contact layer 16 may also be a permeable membrane so as to allow medications, liquids, and/or gasses to flow from within the dome 28 and through the skin contact layer 16 and onto a wound. For example, a medication may flow through the skin contact layer 16 to provide antibiotics, antibacterials, or other medications to a wound. The film used to construct the skin contact layer 16 may also be impregnated with antibiotics, antibacterials, or other medications that may leach out of the film during use to provide treatment for a wound.

An acceptable polyurethane film for use as the skin contact layer 16 is available from Avery Dennison Medical, of Mentor, Ohio, in such a thickness, together with a non-sensitizing, pressure sensitive, acrylic copolymer adhesive coating on one face of the film and ready for use as the adhesive layer 18, and with a silicone coated kraft paper liner already adhered to the layer 18 of adhesive material. Such a laminated material is available from Avery Dennison Medical as its MED 5042 polyurethane film.

The bandage 14 in use thus has a thickness (excluding the liner 20) of not more than about 0.15 mm (6 mils), so as to avoid aggravating pressure on one's skin by added bulk, as inside a shoe, for example.

For still thinner bandages, one may use materials chosen to have a low coefficient of friction between the dome top layer 36 and the skin contact layer 16, or may include a small amount of suitable lubricant (not shown) between the dome top layer 36 and the skin contact layer 16. In this configuration, the dome base layer 38 might be omitted, although this may require a compromise with respect to either the ability of the dome 28 to move easily with respect to the skin contact layer 16, or to the ability of the skin contact layer 16 to conform to the user.

The bandage 14 may be perforated and/or constructed of a permeable material to aid in transmission of vapor and moisture from the skin to which the bandage 14 may be attached. Such perforation and/or permeability may also enhance the flexibility of the skin contact layer and its ability to stretch. Arrays of perforations 80 are preferably present within the opposite end portions 48 and 50 and within the skirt portion 42, but perforations may or may not be present in the dome 28 itself. Perforations 80 are preferably separated by only a small spacing 81, such as 1.27 mm (0.050 in.), and preferably in the range of 0.76-2.28 mm (0.030-0.090 in). An area 76 extending along each of the straight margins 58 of the dome top layer 36 is preferably free from perforations, in order to avoid interference with the stability of adhesive attachment of the dome 28 to the skin contact layer 16. For example, an area 76 extending for a distance 78 of about 1.6 mm (0.0625 in) on either side of each of the straight margins 58 is left free from perforations 80 in the bandage 14 described herein above.

The bandage 14 can be applied to a person's skin with the dome 28 aligned with a portion of the skin which otherwise might be rubbed by an article interfacing with the person's skin, such as a wheelchair, bedclothing, shoes, athletic equipment, etc. As shown in FIG. 6, the dome 28 is aligned with the prominence on the outside of a person's ankle, and the top portion 30 of the dome 28 is free to move relative to the skirt 42 and the underlying dome base layer 38, while the skin contact layer 16 is securely attached to the person's skin by its adhesive layer 18. The flexibility and elasticity of the skin contact layer 16 permit it to conform easily to the person's ankle, and to stretch and retract itself as the person undergoes activity. The top portion 30 of the dome 28 is free to move in any direction, as indicated by the arrow 66, within the limitations imposed by the height of the dome 28, as previously explained.

The length 24 of the bandage 14, or another bandage of like construction but different size, is intended to permit the opposite ends 48 and 50 of the skin contact layer 16 to overlap one another as shown in FIG. 7 when the bandage is applied to a body part such as a finger or the toe 96, in order to securely hold the bandage in place with the dome 28 located where it is needed to reduce shear and friction forces for prevention or treatment of skin breakdown.

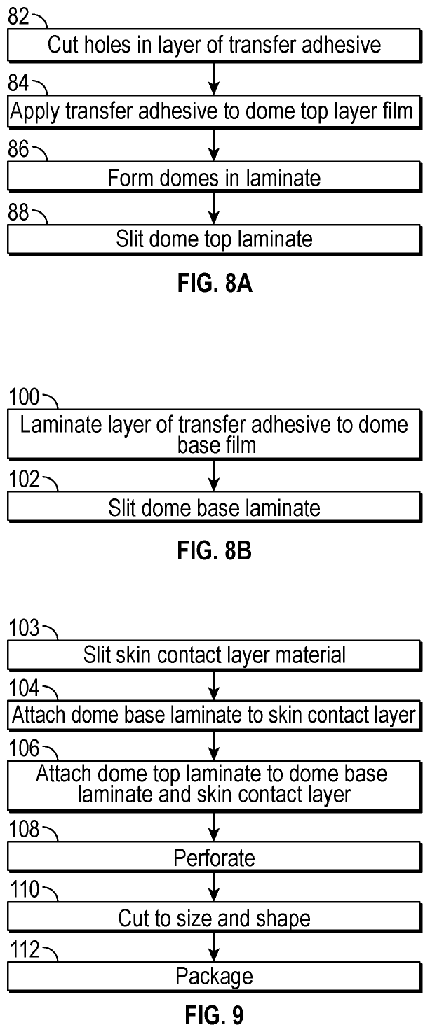

Referring to FIG. 8A, a friction reducing bandage such as the bandage 14 is preferably made using known production equipment for working with films of plastics or other webs of material, performing a novel combination of steps. As a first step, shown at 82 in FIG. 8, domes 28 are prepared by cutting openings corresponding to the size and shape of the dome 28 in a web of a transfer adhesive material to form the adhesive layer 44. The web of transfer adhesive material includes an easily released protective carrier sheet or liner of coated paper on at least one side of a layer of adhesive material. Once the openings have been cut in the layer of transfer adhesive material, the transfer adhesive is adhered to one side of a web of the 1 mil. polyethylene film material for the dome top layer 36, forming a laminate of the dome top layer 36, the layer 44 of adhesive, and the coated paper carrier, as indicated at 84. These first two steps may both be accomplished using a rotary converter apparatus such as, for example, the Crusader® Converter available from Delta Industries of Minneapolis, Minn., or an equivalent converter apparatus capable of unwinding, separating, guiding, combining, and rewinding multiple webs from individual spools while maintaining required alignments and registrations among the various webs.

Thereafter, as noted at 86, a dome 28 is formed in the dome top layer 28 within each opening through the layer 44 of adhesive, using heat and pressure to shape the polyethylene or other polymeric film. The domes 28 may be formed in sequentially produced arrays of several domes 28 produced simultaneously with each application of heat and pressure, using a machine such as is ordinarily used to form considerably thicker sheets of plastics materials to produce clam shell packaging. One such machine which has been found suitable for forming the very thin polyethylene film of the dome top layer 36 to produce an array of domes 28 and their skirts 42 for twenty-seven bandages 14 in each heating and pressing cycle is available from Preco Industries, Inc. of Lenexa, Kans., as its ConvertaForm™ forming system. This machine utilizes appropriate heated molds and dies in an automated air pressure forming system and can handle a forming area up to about 330×420 mm (13 inches by 16.5 inches) with each cycle. For the bandage 14, such domes are prepared using a tool to produce a dome initially having a height 33 of 0.250 in., including a flat top portion 30 with a diameter 31 of 0.900 in., interconnected with the side wall portion 32 by a radiused transition zone 92 whose radius 94 is preferably 0.125 in. but which may satisfactorily be within the range of 1.27-3.81 mm (0.050-0.150 in.). After formation of the domes 28, the polyethylene material typically retracts slightly so that the dome height 33 is ultimately about 4.76 mm (0.1875 in). The domes 28 are thus left free to move in any direction along the dome base layer 38, within the limitations established by the height 34 of the side wall portion 32, the extent of the transition zone 92, and the flexibility of the material of the dome top layer 36.

Instead of the top portion 30 being flat, it may also be formed to extend, for example, in the form of a portion of a spherical surface without adversely affecting the properties of the completed bandage 14.

After the domes 28 have been formed in the laminate of polyethylene dome top layer 36 and layer 44 of transfer adhesive material, the laminate is slit, as mentioned at 88, to form a continuous web of the laminate of the dome top layer 36 with domes 28 formed therein, the adhesive layer 44, and its cover sheet (not shown), bounded by the straight margins 58, and with the width 72 previously mentioned.

Referring next to FIG. 8B, using a rotary converter such as that previously mentioned, a layer of transfer adhesive to form adhesive layer 40 is laminated to a web of film material to be used as the dome base layer 38 as shown at 100. As shown at 102 the laminate of the dome base layer film and transfer adhesive is slit to form straight margins 52 separated by the required width 70.

As shown in FIG. 9, the bandages are assembled using a rotary converter such as the one described previously. First, a web of the required material for the skin contact layer 16 is slit to a width such as about 76 mm (3 in), wide enough to encompass the length 24 of the bandage 14, as indicated at 103.

Next, the dome base laminate of layers 36 and 40 formed as mentioned above at 102 is applied to the skin contact layer 16 by appropriately removing any carrier or liner from the layer 40 of transfer adhesive material, and the dome base layer 38 is placed on the skin contact layer 16 and fastened to it by the action of the adhesive layer 40, as shown at 104. The dome base layer 38 is aligned with a middle part of the skin contact layer web, so that its straight margins 52 face toward the sides of the slit web of skin contact layer material and are parallel with the length of that web of skin contact layer material.

Next, as noted at 106, the previously fabricated dome top laminate of domes 28, skirt portions 42, and adhesive layer 44 is attached to the dome base layer 38 and the skin contact layer 36 in proper alignment with the dome base layer 38, so that the straight margins 58 of the dome top layer 36 are located outside the straight margins 52 of the dome base layer and parallel with them. The skirt portion 42 of the dome top layer 36 is thus attached by the adhesive layer 44 both to the dome base layer 38 and to the carrier layer 16, where the margins 58 of the dome top layer 36 extend beyond the straight margins 52 of the dome base layer 38.

After the dome top layer 36 is attached to the dome base layer 38 and skin contact layer 16, each bandage 14 is appropriately perforated, 108, preferably by a sonic perforating machine associated with the converter. For example, a suitable sonic perforator is available from Branson Ultrasonic Corporation, of Danbury, Conn. The individual bandages 14 are then cut to finished size and shape, and the cut 22 is made in the liner 20, as shown at 110, preferably by die cutting, using the rotary converter. Alternatively, the liner 20 can be removed and replaced by a combination of liner portions of which one includes a margin folded back as a flap 23, shown in broken line in FIG. 1, before the individual bandages 14 are cut to finished size and shape.

The bandages 14 are then released from the surrounding areas of the laminated webs of skin contact layer 16, dome base layer 38, and dome top layer 36, and as noted at 112 the individual bandages are appropriately packaged.

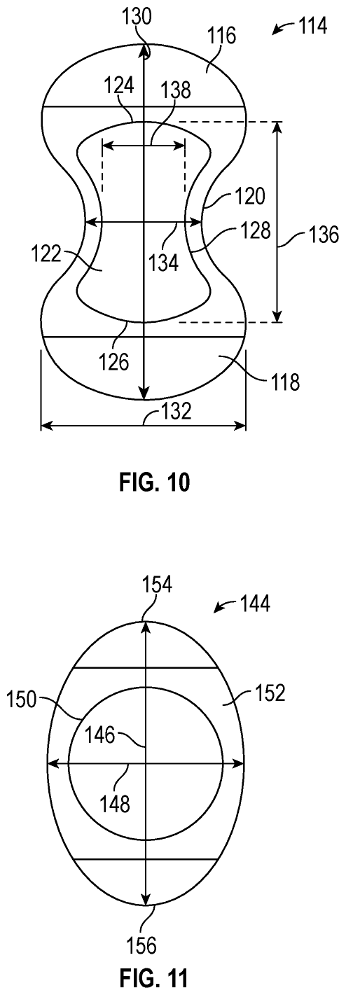

Referring next to FIG. 10, a bandage 114 is of similar construction to the bandage 14, but is of a different shape, resembling a figure-of-eight having a pair of enlarged end portions 116 and 118 and a narrow waist portion 120. A dome 122 of the bandage 114 also has a pair of convexly arcuate end portions 124 and 126 that are interconnected by a narrower waist portion 128. Such a bandage 114 is intended principally for use to protect convex portions of one's anatomy, such as elbows, heels, fingers, and toes, and can be made in a range of sizes, with a preferred overall length 130 of 57 mm (2.25 in.) and an overall width 132 of 31.75 mm (1.25 in.), with a waist width 134 of 22.23 mm (0.875 in.). The overall length 130 and overall width 132 could be varied within a range of at least ±½ in.

The dome 122 is significantly shorter than the overall length 130 to provide ample adhesive-carrying area in the end portions 124 and 126 of the skin contact layer, to attach the bandage securely to a person's skin. The dome 122 thus has a preferred overall length 136 of about 35 mm (1.375 in.) and an overall width 138 of about 12.7 mm (0.5 in.), although both of these dimensions could be varied within a range of at least ±6.3 mm (0.25 in.). The height of such a dome could be within the range of 4-6 mm (0.18-0.25 in.).

Referring next to FIG. 11, a bandage 144 has an oval shape and may be elliptical, for example, with an overall length 146 of 108 mm (4.25 in.) and an overall width 148 of 76.2 mm (3 in.), both of which could be varied by as much as ±19 mm (±0.75 in.). A centrally located circular dome 150, whose diameter is preferably about 57.2 mm (2.25 in.), or could be varied within ±9.5 mm (±% in.) of that size, is provided to correspond with the length 146 and width 148 of the bandage 144. The bandage 144 has an amply wide skirt portion 152 surrounding the dome 150 and also has some extension of a suitably flexible and elastic skin contact layer at each of the ends 154 and 156. The larger size of the bandage 144 by comparison with the previously described bandages 14 and 114 makes the bandage 144 more appropriate for use in prevention and treatment of pressure ulcers. The bandage 144 is preferably manufactured of film materials and adhesives such as those described above, or of variations of those materials having a different moisture vapor transference rate. Such a bandage could be used in locations such as a person's sacrum (lower back), greater trochanter (hip), gluteus maximus (buttocks), heels, or elbows.

The bandage could also be produced with the dome and skin contact layer in other shapes to be used in other particular applications, without departing from the invention.

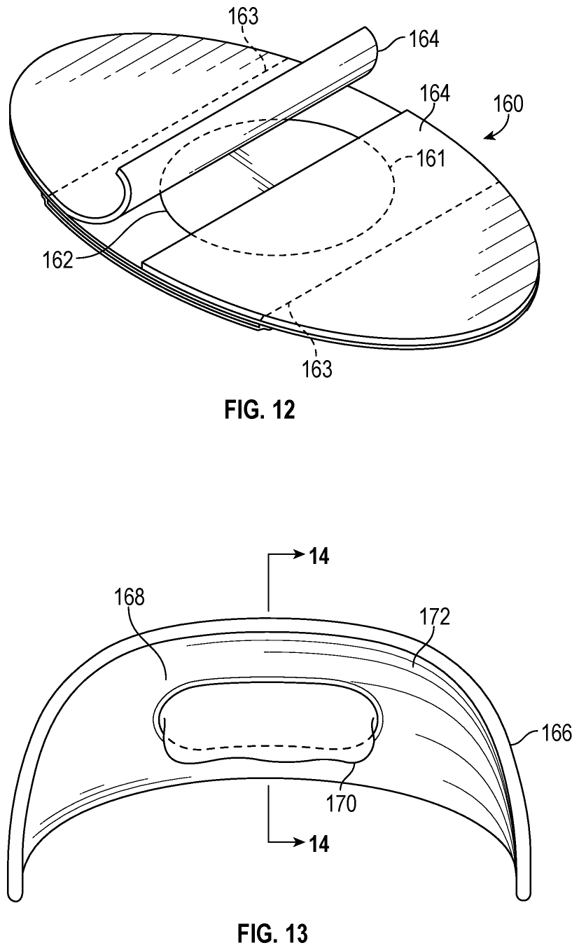

The size of the bandage 14 could also be varied, keeping the same proportions to provide a bandage 14 whose size is, for example, 20 percent larger or 40 percent larger than previously mentioned, in order to protect an area of a patient's skin with an appropriately large bandage.

Where skin that is already blistered or abraded is to be treated to enhance healing of such skin, a bandage 160 shown in FIG. 12, may be utilized. Although the bandage 160 may generally be similar to the bandage 14, in an area 161 corresponding generally in size and shape to the dome, and optionally including the area 163 of the skirt portion of the dome top layer and the area of the dome base layer, the adhesive carrying lower or second side of the skin contact layer may be left without a layer of adhesive, or may be provided with a layer 162 of an adhesive translucent, flexible hydrocolloid material. Such a hydrocolloid material, once the liner 164 has been removed from the bandage, is placed in contact with the patient's sore or abraded skin or other wound and can absorb wound exudate, promote a moist wound-healing environment, and provide cushioning. Such a hydrocolloid material is available, for example, from Avery Dennison Medical, of Mentor, Ohio, as its MED 2190H, an 18 mil hydrocolloid, low peel force, transfer adhesive tape. Similar material may also be used in various thicknesses in a range of 10 to 50 mils for various applications.

A device embodying the present invention could also be applied to a surface of a device or an article of clothing, rather than to the user's skin. For example, a layer of a suitable material corresponding to the skin-contact layer 16 could be attached by sewing, or other bonding technique, i.e., heat or ultrasound, to the inside of a user's apparel, i.e., underwear, sock or shoe. It could also be attached to the inside of a shoulder pad, elbow pad or backpack strap, etc., for use in athletic or recreational activities.

In order to deal with other possible circumstances, friction reducing devices embodying the present invention could be incorporated into many articles during original manufacture of such articles, such as hospital mattress covers, wheelchair cushions, bicycle seats, shoe inserts, removable heel counters for shoes, other locations in the interior of shoes, socks, undergarments, straps for undergarments, and backpacks.

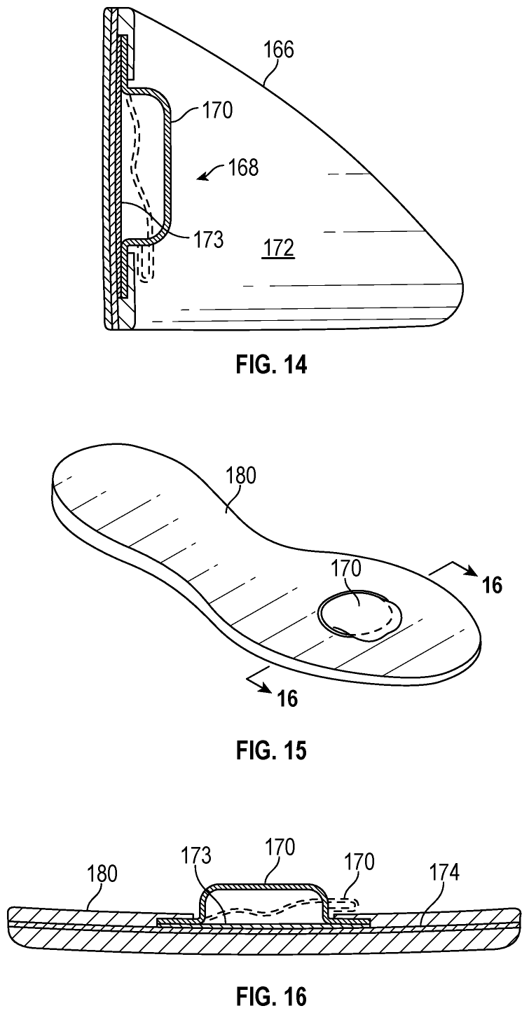

For example, FIGS. 13 and 14 show a removable heel counter 166 for a sports shoe, incorporating a friction reducing device 168 according to the present invention in which a dome 170 is exposed on an inward-facing surface 172 of the removable heel counter 166. The friction reducing device 168 may also include a dome base layer 173, between the dome 170 and a dome supporting layer 174. The dome base layer 173 would be attached to the dome supporting layer 174 and be of material over which the dome 170 can glide freely. The friction reducing device 168 thus incorporates structures capable of performing the function of a friction reducing bandage 14, as described above, in the supporting structure of a heel counter 166, which may be a removable shoe accessory or a permanent part of a shoe.

The dome supporting layer 174 of such a friction reducing device 168 corresponds to the skin contact layer 16 of the bandage shown in FIGS. 1-5 and may be of a thicker material, and may be thermally laminated into the structure of the heel counter 166 itself. If a dome base layer 173 is not included, the dome supporting layer 174 should be of material that would not interfere with the gliding motion of the dome top layer 170. Instead of the thin polyurethane film material of the skin contact layer 16, the supporting layer 174 is preferably of a thicker film or may be of a durable, sturdy textile fabric, such as a woven cloth or knitted textile material, or of a different plastic such as a film or sheet of polyamide or polyester compatible with the adjacent material of the rest of the heel counter 166, since greater durability than for a bandage is preferred.

In a different article such as a replaceable accessory insole 180, shown in FIGS. 15 and 16, the supporting layer 174 could also be of a plastic material such as polyurethane, suitable to be molded into, or inserted between other layers of the structure of the article concerned, to provide a secure incorporation of a friction reducing device 182 according to the present invention. The friction reducing device 182 also includes a dome 170 which is carried by a supporting layer 174 and exposed to be contacted by the user's foot to function in the same way as the dome 28.

The friction reducing domes 170 can be manufactured in generally the same fashion as that described above with respect to the bandage 14, with modifications as necessary to the supporting layer 174 depending on the article in which the friction reducing device is being incorporated.

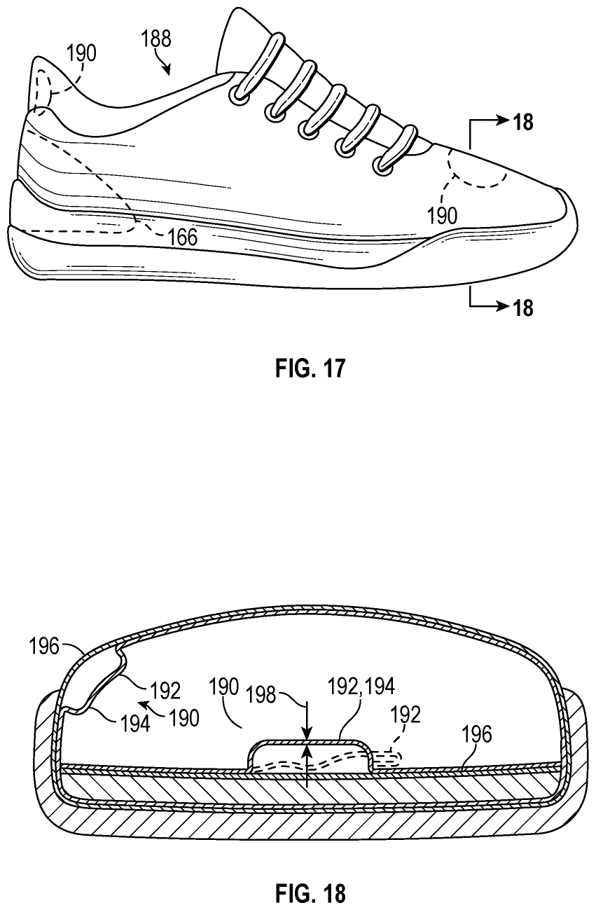

As shown in FIGS. 17 and 18, as an example of including a similar friction-reducing device in an article of sports equipment or other application such as those mentioned above, a shoe 188 may incorporate friction-reducing devices 190 each including an integral dome 192 that functions in generally the same way as the dome 28. The friction reducing device 190 may be located in a permanently included heel counter portion of the shoe, in a heel collar, in the permanent insole, or at another critical place in the upper of such a shoe 188.

In order to ensure reasonably long durability of the friction-reducing device 190 incorporated in an article of clothing or sports or other equipment such as the shoe 188, a dome top layer 194 and dome base layer 196 may be made of a flexible material of greater strength than the 25 microns-thick polyethylene film used as the dome top layer 36 of the bandage 14 described above. For example, a polyethylene film material having a greater thickness 198, in the range of 25 microns to 1.525 mm (1-60 mils) would be satisfactory. Instead of polyethylene, another polymeric material such as a polyester film or a soft and closely woven or knitted textile fabric might be used, although it might require the use of a small amount of a lubricant between the dome top layer 194 and the dome base layer 196. More preferably, the top layer 194 and bottom layer 196 are appropriately formed and interconnected portions of the textile fabric, plastic-impregnated fabric, or plastic film of which the shoe 188 or other article is constructed, thin enough to be easily flexible, and lubricated as necessary to be able to slip readily along each other. Such formation and interconnection of the layers of material to form the domes 192 is performed as part of the process of assembling the article including the domes 192.

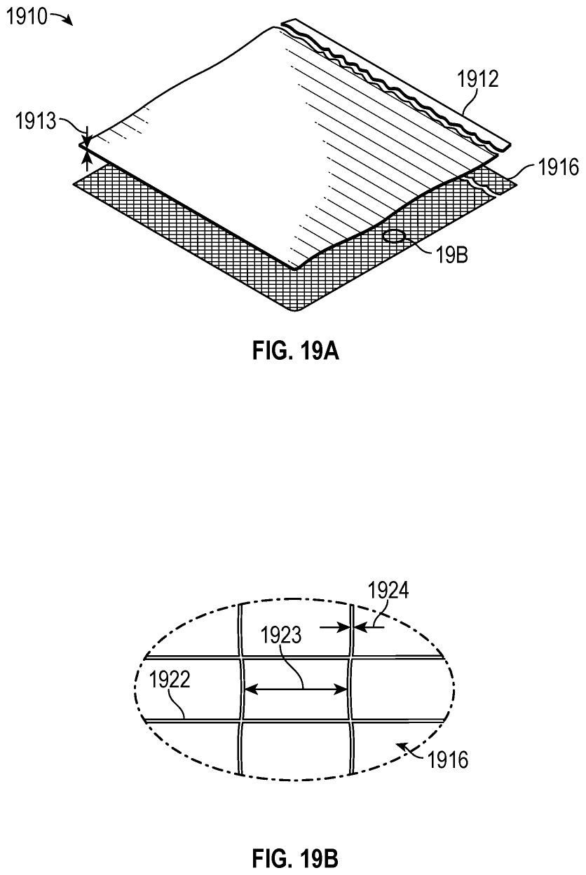

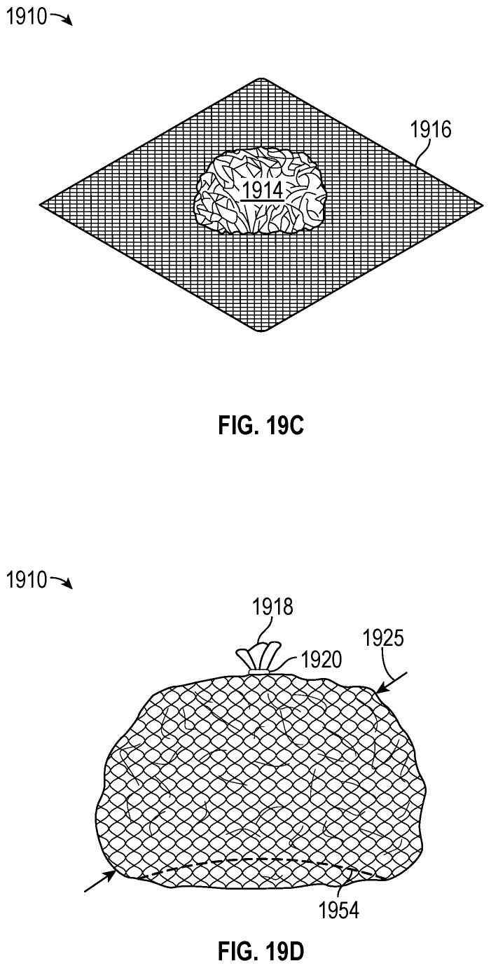

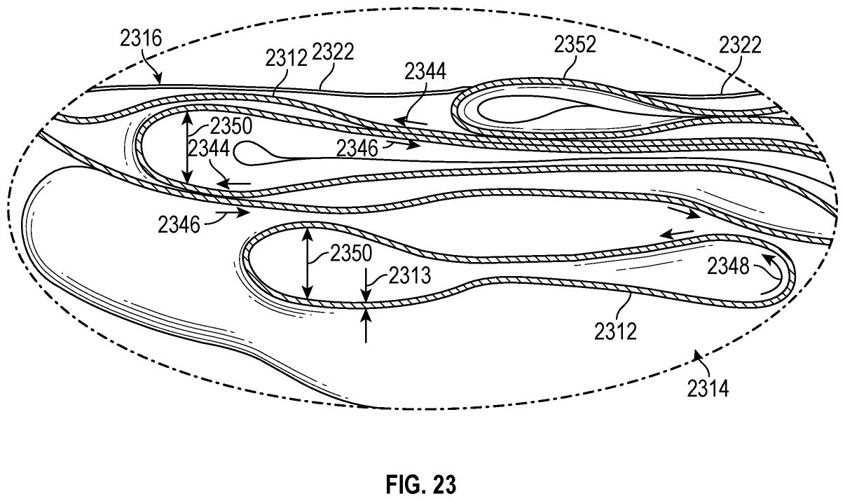

FIGS. 19A-19D illustrate one method of assembling a comfort insert 1910 for use within a ‘person’s footwear. Construction may begin with a sheet of a flexible thin plastic film 1912, as shown in FIG. 19A. The plastic film 1912 is preferably very thin, with a thickness 1913 illustrated in FIG. 23 that is preferably less than 0.003 inch and may, for example, be about 0.0005 inch. The film material 1912 should have a low coefficient of friction, and may, for example, be polyethylene film having a thickness 1913 of 0.0005 inch. Such film material is commonly available and used by laundries and dry cleaners to protect apparel that has been laundered or dry cleaned. Such film commonly has a static coefficient of friction in the range of 0.25 or below. The film material 1912 may, alternatively, be polyurethane film of a similar thickness, also widely available, but often having a somewhat higher, yet still low, static coefficient of friction. The coefficient of friction of such polyurethane film may preferably be in the range of 0.5 or below. Other plastic film materials may also be suitable if they are sufficiently flexible yet resilient and have a sufficiently low coefficient of friction to perform as explained below.

The thickness 1913 of the film material 1912 is preferably small, in the range of 0.0003-0.003 inch, so that several layers of the film material 1912 will still amount to no more than the thickness of a person's stocking or sock. Using such thin film material 1912 will give the result that the comfort insert 1910 will not easily be felt and noticed as a foreign object present within the footwear in which the comfort insert 1910 is being used. A further reason for the film material 1912 to be thin is to ensure that it will be flexible enough so that several layers of the film are free to move with respect to each other, with folds of the film easily rolling and allowing the layers of film on either side of a fold to slip along each other.

At the same time, some thickness and a small amount of resiliency, or springiness, in the film and possibly some air trapped between the folds of the film may be desired, as it can result in the comfort insert 1910 being able to provide a small amount of cushioning. In that respect, a film 1912 of polyethylene having a thickness 1913 of 0.002 inch can provide some noticeable cushioning. The comfort insert could be made using film 1912 of polyethylene of a thickness 1913 within the range of 0.0005-0.003 inch to provide a selected amount of resiliency and cushioning. A thickness 1913 of polyethylene film of about 0.001 inch has been found to be a very good compromise with ample flexibility yet some useful resiliency.

As shown in FIGS. 19A and 19C, in making one preferred embodiment of the comfort insert 1910 a single sheet of film 1912, for example a sheet of polyethylene, whose thickness 1913 is 0.001 inch and whose area is about 1 square yard, is crumpled or wadded. The film 1912 is thus wrinkled and folded to some extent incidentally and randomly folded, to form a loose, rounded, ball-like mass to serve as the friction-reducing, shear-reducing body 1914 of the comfort insert 1910, as shown in FIG. 19C.

Assembly of this embodiment of the comfort insert 1910 proceeds further with wrapping piece of a material such as an area of an open-mesh netting material 1916, formed of very fine filament, as shown in FIGS. 19A, 19B, and 19C, around the body 1914 and fastening the netting material to form an enclosing or containment element 1918 surrounding the body 1914. The material 1916 of the containment element 1918 should be flexible and permeable to fluid. Preferably the material 1916 of the containment element 1918 is open enough to expose areas of the flexible film material 1912, and so an open mesh netting is particularly suitable. A sufficiently strong thin film or other thin, flexible sheet material could also be used as the containment element 1918 so long as it is capable of containing the body 1914 despite forces that may be applied to the comfort insert 1910 when it is in use. Such a film used as the containment element 1918 should preferably be perforated to generously expose areas of the film material 1912 of the body 1914. Alternatively, or additionally, such film material should itself be somewhat slippery.

Within the body 1914 the thin film material 1912 is simply crumpled and wadded into a loose ball shape. Preferably the thin film material 1912 thus includes many loosely formed folds and several overlying individual areas whose surfaces confront one another, as shown, for example, in FIG. 23. The netting or other highly pervious or perforated material 1916 is then gathered around the body 1914 to form the containment element 1918 as by, for example, tying a suitably strong yet fine filament or thread 1920 around the gathered material 1916 to contain and enclose the body 1914. Because the filaments defining the mesh of the netting material 1916 are very fine the bunched netting, even when securely tied, still forms only a very small lump, that is unlikely to be intrusive when the comfort insert 1910 is in place, particularly if the tied lump of netting is kept to one side when the comfort insert 1910 is placed into a person's footwear for use.

As shown in FIG. 19B, the material 1916 of the containment element 1918 in one preferred embodiment of the comfort insert 1910 may be a netting material of fine filaments 1922 forming an open mesh with generally square mesh openings having a mesh opening dimension 1923 in the range of 0.20 inch-0.75 inch, for example, preferably about % inch. Netting material found to be satisfactory for use as the material 1916 of the containment element 1918 is widely available and used as hair netting by food service personnel, for example. Individual filaments of sufficient strength for forming the meshes of such netting may be of a flexible material such as a polyester or a polyimide, for example. The filaments forming the mesh may have, for example, diameters 1924 of 0.0005-0.004 inch and preferably in the range of 0.002 or 0.003 inch. Since the filaments 1922 forming the meshes are of very small diameter, the exposed area of the thin plastic film material 1912 of the body 1914 is far greater than the combined area of filaments of such netting material 1916 of the containment element 1918, and so a majority of the exterior of the comfort insert 1910 is low-friction surfaces of the flexible thin-film material 1912 of the body 1914, exposed through the containment element 1918. As an additional means of securing the crumpled and folded thin plastic film material 1912 into a unified body 1914 a very fine filament or thread (not shown) may be sewn loosely through the entire ball or body 1914 either before or after the netting 1916 or other pervious material is wrapped around it as the containment element 1918. This additional means may help the containment element 1918 and the comfort insert 1910 in general, to retain a desired form or shape.

The comfort insert 1910 when thus completed need not have any particular shape, but may initially be in a generally spherical shape as a ball having a diameter 1925 in the range of 2 inches-5 inches, preferably in the range of 3 inches-4 inches. The body 1914 is loosely formed, and so the portion of the comfort insert 1910 beneath a person's foot can easily be compressed into a flat and relatively thin configuration within a user's shoe or other footwear.

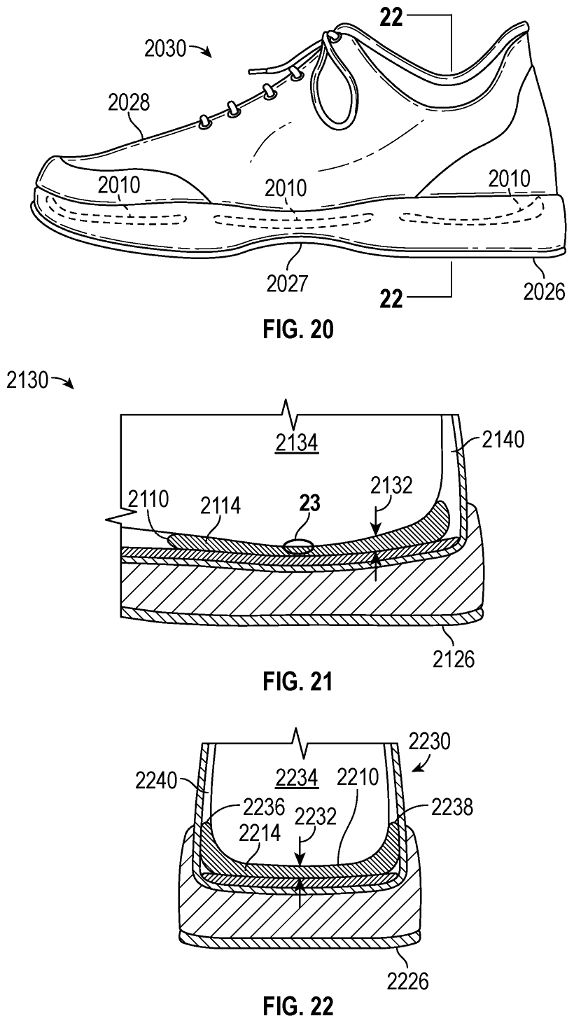

The comfort insert 1910 may be placed in any desired location within a person's shoe to provide support and reduce friction. For example, as shown in FIG. 20, comfort inserts 1910 are shown in place in a heel portion 2026, an arch portion 2027, or a toe box portion 2028 of the shoe 2030. In each location where the comfort insert 1910 is placed, the user's weight squeezes at least a main portion of the comfort insert 1910 down to a relatively very thin vertical dimension or thickness 2132 immediately under the user's foot 2134, while outer margins 2236 and 2238 of the comfort insert 1910 may remain less compressed and serve to fill otherwise unoccupied space 2140 within the shoe 2030, as shown in FIGS. 20, 21, and 22.

As shown best in FIG. 23, the crumpled film material 1912 of the body 1914 of the comfort insert 1910 includes several layers of the film material 1912 that are free to slip and slide along one another's surfaces, as in the directions relative to each other indicated by the arrows 2344 and 2346. Because of the low coefficient of friction of the thin film material 1912, as well as a quantity of air or other lubricant material that may be trapped between the thin layers, the effective amount of friction between the user's foot and the insole of the shoe is minimized. The lubricant material could be an inert non-viscous liquid or a lubricant powder, such as powdered PTFE, for example, which can reduce the coefficient of friction significantly below that of the bare thin plastic film 1912. The comfort insert 1910 minimizes the amount of shear stress applied to the skin of the user's foot as the foot moves in all planes within any loose space 2140 available within the shoe 2030. Even though there may be multiple layers of the film material 1912 in the body 1914 of the comfort insert 1910, because the thickness 1913 of each layer is so thin, the total thickness of the comfort insert 1910 where it is fully compressed is similar to the thickness of an ordinary men's sock and is thus barely noticeable, if at all.

As indicated by the arrow 2348, when various layers of the film material 1912 slide with respect to one another the film material 1912 may roll through the folds and thus move the folds within the body 1914. Depending on the amount of resiliency of the thin film material 1912, and partly depending upon the thickness 1913, the body 1914 will have some overall resiliency, tending to oppose compression of the body 1914 as indicated by the arrow 2350, and thus can provide some cushioning for the user's foot. As illustrated in FIG. 23, surfaces of the thin film material 1912 may bulge outward through the mesh of a containment member 1918 of netting material, as at 2352, enabling the comfort insert 1910 to move within the user's shoe and thus adjust its position during use.

The comfort insert 1910 as described above ordinarily will remain generally where desired within an enclosed shoe. However, when used in open shoes such as women's dress shoes or sandals, it may be desired to provide some additional security to prevent the comfort insert 1910 from moving away from a desired position within or on a person's footwear. For that purpose, an adhesive material compatible with the thin film material 1912 may be applied to the comfort insert 1910 after it is assembled, as by spraying a small amount of an adhesive or a nonslip material onto an area 2354 of the comfort insert 1910, shown in FIG. 19D. By placing the comfort insert 1910 with the area 2354 of adhesive or nonslip material in contact with the insole or exposed upper surface of the shoe or sandal, the comfort insert may be retained in a desired location with respect to the footwear during use.

While assembly of the comfort insert 1910 as described above may be accomplished simply, a somewhat different but generally similar comfort insert may be constructed by stacking and enclosing a number of smaller sheets of thin plastic film material 1912 having alow coefficient of friction so that they can slide over one another. The multiple sheets of thin plastic film material may be contained between a pair of flat pieces of perforated film material or open mesh netting material 1916 interconnected with each other along the peripheries of the material 1916. While such a comfort insert as just described can provide the benefits of reducing friction as a result of including multiple layers of thin plastic film material, such construction may provide a lesser amount of cushioning, unless at least some of the smaller sheets of thin plastic film material include folds.

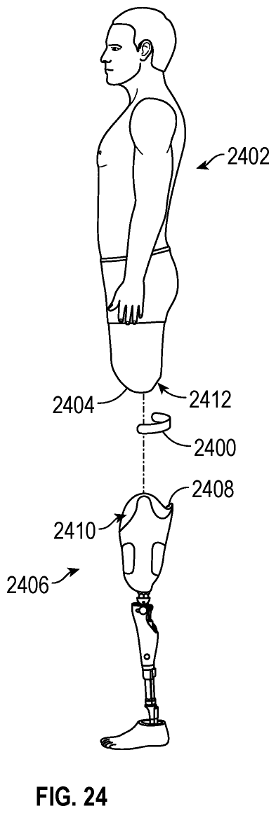

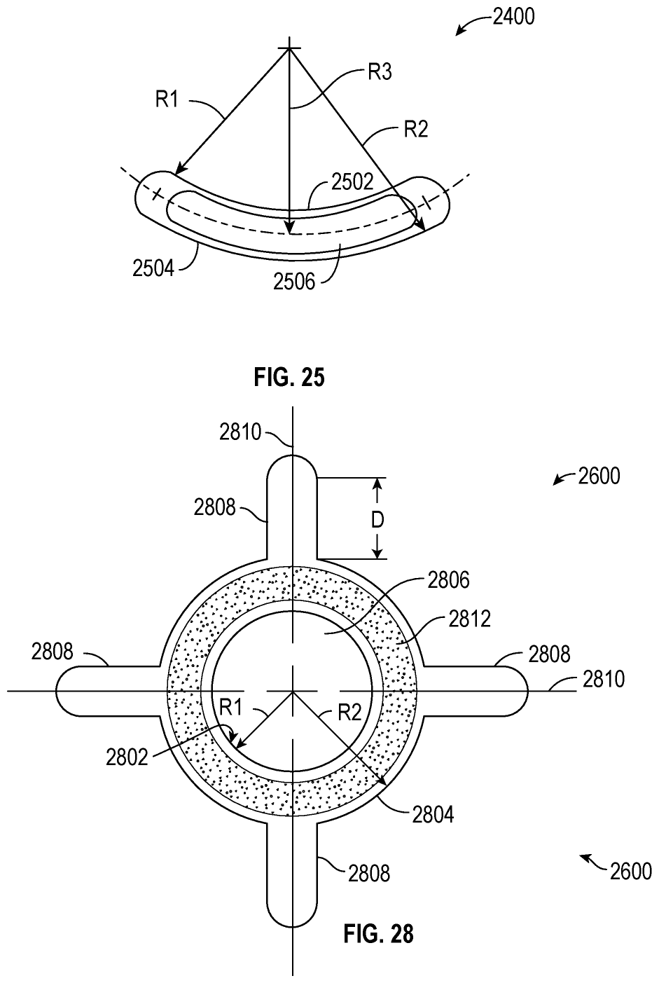

FIG. 24 is a view a bandage 2400 and use thereof in accordance with at least one embodiment of this disclosure. FIG. 25 is a plan view of bandage 2400 in FIG. 24 in accordance with at least one embodiment of this disclosure. As disclosed herein, a patient 2402 may have an amputated appendage, such as a transfemoral amputation as shown in FIG. 24. The amputation may leave patient 2402 with a residual limb 2404. As part of treatment, a prosthetic 2406 may be fitted to residual limb 2404. Prosthetic 2406 may include a hard socket 2408.

Hard socket 2408 may rub against residual limb 2404 and cause pain, discomfort, and/or other edema for patient 2402. To relieve or otherwise minimize pain and/or discomfort, bandage 2400 may be placed inside hard socket 2408 to provide padding or other friction reduction between a surface 2408 of hard socket 2408 and a surface 2412 of residual limb 2404.

As disclosed herein, bandage 2400 may be flexible and positionable around body parts, such as residual limb 2404. Bandage 2400 may be curved. For example, a first edge 2502 of bandage 2400 may have a first radius, R₁, a second edge 2504 of bandage 2400 may have a second radius R₂. The first and second radii can be constant or vary depending on the prosthetic and/or residual limb to which bandage 2400 is fitted. For example, for a transfemoral amputation as shown in FIG. 24, first and second radii may be constant. For a choppart amputation, first and second radii may be different to allow bandage 2400 to conform to residual limb of a foot.

As further disclosed herein, bandage 2400 may include films, lubricants, etc. as disclosed above that allow surfaces of bandage 2400 to move relative to one another to reduce friction and provide relief for patient 2402. For instance, bandage 2400 may include a pocket 2506 that includes one of more lubricants, films, etc. as disclosed above reduce friction between residual limb 2404 and hard socket 2408.

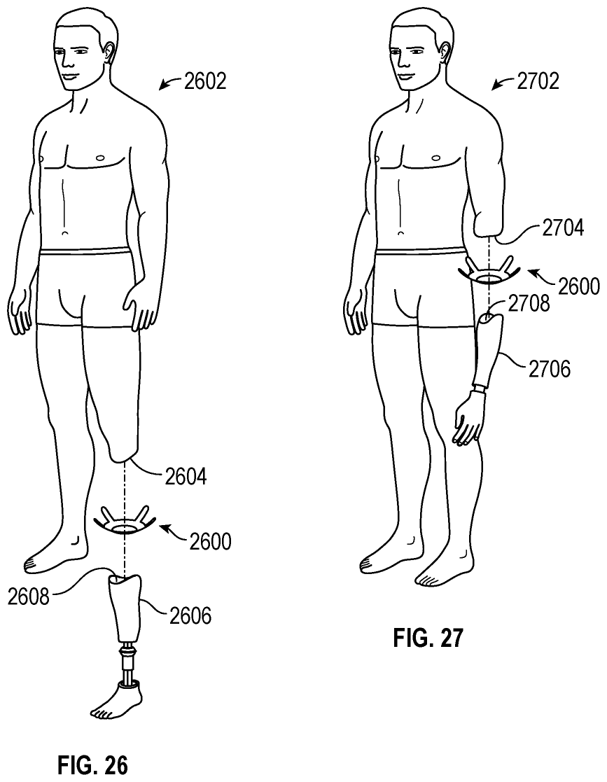

FIG. 26 is a view a bandage 2600 and use thereof in accordance with at least one embodiment of this disclosure. As disclosed herein, a patient 2602 may have an amputated appendage, such as a transfemoral amputation. The amputation may leave patient 2602 with a residual limb 2604. As part of treatment, a prosthetic 2606 may be fitted to residual limb 2604. Prosthetic 2606 may include a hard socket 2608.

Hard socket 2608 may rub against residual limb 2604 and cause pain, discomfort, and/or other edema for patient 2602 as disclosed herein. To relieve or otherwise minimize pain and/or discomfort, bandage 2600 may be placed inside hard socket 2608 to provide padding or other friction reduction between hard socket 2608 and residual limb 2604.

FIG. 27 is a view a bandage 2700 and use thereof in accordance with at least one embodiment of this disclosure. As disclosed herein, a patient 2702 may have an amputated appendage, such as a transradial amputation. The amputation may leave patient 2702 with a residual limb 2704. As part of treatment, a prosthetic 2706 may be fitted to residual limb 2704. Prosthetic 2706 may include a hard socket 2708.

Hard socket 2708 may rub against residual limb 2704 and cause pain, discomfort, and/or other edema for patient 2702 as disclosed herein. To relieve or otherwise minimize pain and/or discomfort, bandage 2700 may be placed inside hard socket 2708 to provide padding or other friction reduction between hard socket 2708 and residual limb 2704.

FIG. 28 is a plan view of bandage 2600 in FIGS. 26 and 27 in accordance with at least one embodiment of this disclosure. As disclosed herein, bandage 2600 may be flexible and positionable around body parts, such as residual limb 2604 and/or 2704. Bandage 2600 may be circular as shown in FIG. 28. For example, a first edge 2802 of bandage 2600 may have a first radius, R₁, a second edge 2804 of bandage 2600 may have a second radius R₂. The first and second radii can be constant or vary depending on the prosthetic and/or residual limb to which bandage 2600 is fitted. For example, for a transradial amputation as shown in FIG. 27, first and second radii may be constant to accommodate a relatively symmetric residual limb 2704. For a disarticulation amputation, first and second radii may be different and bandage 2600 may have an oval and/or ovoid shape to allow bandage 2600 to conform to residual limb that is not symmetrical and or otherwise has an irregularity.

As disclosed herein, bandage 2600 may have a central opening 2806. Opening 2806 may allow portions of residual limbs 2604 and 2704 to pass therethrough. Bandage 2600 may also include one or more protrusions 2808. Protrusions 2808 may extend from edge 2804 a distance D. Distance D may be the same or different for each of protrusions 2808. Thus, while FIG. 28 shows bandage 2600 as being symmetric about axes 2810, bandage 2600 may be asymmetric about any one of axes 2810.

As further disclosed herein, bandage 2600 may include films, lubricants, etc. as disclosed above that allow surfaces of bandage 2600 to move relative to one another to reduce friction and provide relief for patient 2402. For instance, bandage 2600 may include a pocket 2812 that includes one of more lubricants, films, etc. as disclosed above reduce friction between residual limb 2404 and hard socket 2408.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

### Additional Notes

The following, non-limiting examples, detail certain aspects of the present subject matter to solve the challenges and provide the benefits discussed herein, among others.

Example 1 is a protective bandage comprising: a first flexible film forming a contact layer having a first side opposite a second side; a second flexible film forming a dome attached to and extending over a portion of the first side of the contact layer, the dome having a height at least partially defined by a side wall and a top portion of the second flexible film, the contact layer extending beneath the dome and the top portion being freely movable along the first side of the contact layer through a distance related to the height; and a shear-reducing element located inside the dome, wherein the protective bandage is sized to fit in between a skin surface to be protected and an interior surface of a garment worn by a user.

In Example 2, the subject matter of Example 1 optionally includes wherein the shear-reducing element includes a medication and the first flexible film is a permeable membrane that allows the medication to flow from the dome onto the skin surface to be protected.

In Example 3, the subject matter of any one or more of Examples 1-2 optionally include wherein the shear-reducing element includes a plurality of layers comprising a thin plastic film material, the thin plastic film material folded to form the plurality of layers.

In Example 4, the subject matter of Example 3 optionally includes wherein the shear-reducing element includes a lubricant located in between the plurality of layers.

In Example 5, the subject matter of any one or more of Examples 3-4 optionally include wherein the shear-reducing element includes a quantity of air in between the plurality of layers.

In Example 6, the subject matter of any one or more of Examples 1-5 optionally include wherein the shear-reducing element includes a lubricant.

In Example 7, the subject matter of any one or more of Examples 1-6 optionally include wherein the second flexible material comprises an open mesh netting material.

In Example 8, the subject matter of any one or more of Examples 1-7 optionally include wherein the second flexible material includes an exterior portion including a nonslip surface.

Example 9 is a comfort insert for use in footwear, the comfort insert comprising: a skin contact layer made of a first flexible film having a first side opposite a second side, the second side being adhesively attachable to a surface intended to be protected; a hollow dome made of a second flexible film attached to and extending over a portion of the first side of the skin contact layer, the hollow dome having a height at least partially defined by a side wall and a top portion of the second flexible film, the skin contact layer extending beneath all of the hollow dome and the top portion being freely movable along the first side of the skin contact layer through a distance related to the height; and a shear-reducing body element including a plurality of layers comprised of a thin plastic film material, the thin plastic film material folded to form the plurality of layers, wherein the comfort insert is sized to fit in between the surface to be protected and an interior surface of the footwear.

In Example 10, the subject matter of Example 9 optionally includes wherein the shear-reducing element further includes a medication and the first flexible film is a permeable membrane that allows the medication to flow from the hollow dome onto the skin surface to be protected.

In Example 11, the subject matter of any one or more of Examples 9-10 optionally include wherein the shear-reducing element further includes a lubricant located in between the plurality of layers.

In Example 12, the subject matter of any one or more of Examples 9-11 optionally include wherein the shear-reducing element further includes a quantity of air in between the plurality of layers.

In Example 13, the subject matter of any one or more of Examples 9-12 optionally include wherein the second flexible material comprises an open mesh netting material.

Example 14 is a method for making an insert for a shoe, the method comprising: shaping a first flexible film to form a flexible dome including a flexible side wall and a top portion, leaving the flexible dome surrounded by a generally planar skirt of the first flexible film extending radially outward from the flexible dome; crumpling a sheet of a second plastic material into a body including a plurality of folds and a plurality of overlying and mutually confronting surface areas of the second plastic material; placing the sheet of the second plastic material inside the flexible dome; and attaching the skirt adhesively to a skin contact layer of a third flexible film that extends at least beneath the flexible dome so that the flexible dome is collapsible and the top portion is movable along a portion of the sheet of the second plastic material.

In Example 15, the subject matter of Example 14 optionally includes wherein the first flexible film is a fluid permeable containment material, the method further comprising placing a medication inside the flexible dome so as to allow the medication to flow through the fluid permeable containment material.

In Example 16, the subject matter of any one or more of Examples 14-15 optionally include placing a lubricant within the flexible dome.

In Example 17, the subject matter of any one or more of Examples 14-16 optionally include applying an adhesive to the skin contact layer.

In Example 18, the subject matter of any one or more of Examples 14-17 optionally include forming a least one perforation in at least one of the flexible dome and the skin contact layer.

In Example 19, the subject matter of any one or more of Examples 14-18 optionally include wherein the sheet of the second plastic material forms a crumpled ball.

In Example 20, the subject matter of any one or more of Examples 14-19 optionally include the first flexible material having a static coefficient of friction not greater than 0.25.

In Example 21, the apparatuses or method of any one or any combination of Examples 1-20 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A protective bandage comprising:
a first flexible film forming a contact layer having a first side opposite a second side;
a second flexible film forming a dome attached to and extending over a portion of the first side of the contact layer, the dome having a height at least partially defined by a side wall and a top portion of the second flexible film, the contact layer extending beneath the dome and the top portion being freely movable along the first side of the contact layer through a distance related to the height; and
a shear-reducing element located inside the dome,
wherein the protective bandage is sized to fit in between a skin surface to be protected and an interior surface of a garment worn by a user.

2. The protective bandage of claim 1, wherein the shear-reducing element includes a medication and the first flexible film is a permeable membrane that allows the medication to flow from the dome onto the skin surface to be protected.

3. The protective bandage of claim 1, wherein the shear-reducing element includes a plurality of layers comprising a thin plastic film material, the thin plastic film material folded to form the plurality of layers.

4. The protective bandage of claim 3, wherein the shear-reducing element includes a lubricant located in between the plurality of layers.

5. The protective bandage of claim 3, wherein the shear-reducing element includes a quantity of air in between the plurality of layers.

6. The protective bandage of claim 1, wherein the shear-reducing element includes a lubricant.

7. The protective bandage of claim 1, wherein the second flexible material comprises an open mesh netting material.

8. The protective bandage of claim 1, wherein the second flexible material includes an exterior portion including a nonslip surface.

9. A comfort insert for use in footwear, the comfort insert comprising:
a skin contact layer made of a first flexible film having a first side opposite a second side, the second side being adhesively attachable to a surface intended to be protected;
a hollow dome made of a second flexible film attached to and extending over a portion of the first side of the skin contact layer, the hollow dome having a height at least partially defined by a side wall and a top portion of the second flexible film, the skin contact layer extending beneath all of the hollow dome and the top portion being freely movable along the first side of the skin contact layer through a distance related to the height; and
a shear-reducing body element including a plurality of layers comprised of a thin plastic film material, the thin plastic film material folded to form the plurality of layers,
wherein the comfort insert is sized to fit in between the surface to be protected and an interior surface of the footwear.

10. The comfort insert of claim 9, wherein the shear-reducing element further includes a medication and the first flexible film is a permeable membrane that allows the medication to flow from the hollow dome onto the skin surface to be protected.

11. The comfort insert of claim 9, wherein the shear-reducing element further includes a lubricant located in between the plurality of layers.

12. The comfort insert of claim 9, wherein the shear-reducing element further includes a quantity of air in between the plurality of layers.

13. The comfort insert of claim 9, wherein the second flexible material comprises an open mesh netting material.

14. A method for making an insert for a shoe, the method comprising:
shaping a first flexible film to form a flexible dome including a flexible side wall and a top portion, leaving the flexible dome surrounded by a generally planar skirt of the first flexible film extending radially outward from the flexible dome;
crumpling a sheet of a second plastic material into a body including a plurality of folds and a plurality of overlying and mutually confronting surface areas of the second plastic material;
placing the sheet of the second plastic material inside the flexible dome; and
attaching the skirt adhesively to a skin contact layer of a third flexible film that extends at least beneath the flexible dome so that the flexible dome is collapsible and the top portion is movable along a portion of the sheet of the second plastic material.

15. The method of claim 14, wherein the first flexible film is a fluid permeable containment material, the method further comprising placing a medication inside the flexible dome so as to allow the medication to flow through the fluid permeable containment material.

16. The method of claim 14, further comprising placing a lubricant within the flexible dome.

17. The method of claim 14, further comprising applying an adhesive to the skin contact layer.

18. The method of claim 14, further comprising forming a least one perforation in at least one of the flexible dome and the skin contact layer.

19. The method of claim 14, wherein the sheet of the second plastic material forms a crumpled ball.

20. The method of claim 14, wherein the first flexible material has a static coefficient of friction not greater than 0.25.
